(19) United States
(12) Patent Application Publication
(10) Pub. No.: US 2026/0236059 A1
(43) Pub. Date: Aug. 13, 2026

(54) A SYSTEM FOR SYNCHRONISING EXERCISE OR TRAINING MEASUREMENT EQUIPMENT

(71) Applicant: VALD PTY LTD, Newstead, Queensland (AU)

(72) Inventors: Laurie MALONE, Newstead (AU); Samuel JAMES, Newstead (AU); Nishant SRESTHA, Newstead (AU)

(21) Appl. No.: 19/157,580

(22) PCT Filed: Feb. 17, 2023
(86) PCT No.: PCT/AU2023/050107

§ 371 Date: Aug. 18, 2025

(51) Int. Cl. G06F 1/12; A63B 24/00

(52) U.S. Cl. CPC G06F 1/12; A63B 24/0062; A63B 2024/0071; A63B 2220/62

(57) ABSTRACT

A system for synchronising exercise or training measurement equipment such as exercise equipment and a camera. The system includes an electronic device having a processor, at least one piece of exercise equipment, and a camera. The electronic device may be a tablet with an integrated camera. The processor receives measurement data from the exercise equipment and video data from the camera, determines a camera latency, and synchronises the exercise equipment measurement data with the video data using a calculated latency synchronisation modifier.

### FIELD OF THE INVENTION

The invention relates to a synchronisation of different components of exercise or physical training measurement equipment. In particular, the invention relates, but is not limited, to synchronising, including accounting for latency between, force measurement data and video data as an exercise is performed by a user.

### BACKGROUND TO THE INVENTION

Reference to background art herein is not to be construed as an admission that such art constitutes common general knowledge.

It is known to technically measure the performance of an athlete or patient, or the like, performing a type of exercise. Various devices for taking the measurement exist depending upon the nature of the exercise and data of interest. For example, a force measurement platform may be used to measure forces involved during various human movement activities such as jumping or the speed of a human running over a distance may be measured using speed gates.

It's also known to take video recordings of athletes or patients, or the like, performing a type of movement or exercise. Such content can be reviewed by a practitioner in real time and/or by a practitioner and/or the person after the movement or exercise has been performed. Such systems are typically separate with no integration in control or data output.

A user may try to align a specific measurement against a specific point in time in the video, or vice versa, but this is typically a manual process which takes time and is difficult to align accurately. This is exacerbated by the devices having different clocks, recording frequencies (e.g. measurement frequency doesn't align with camera frame rate), and latencies between various devices which is particularly prevalent in wirelessly connected devices.

### OBJECT OF THE INVENTION

It is an aim of this invention to provide a system for synchronising exercise or training measurement equipment which overcomes or ameliorates one or more of the disadvantages or problems described above, or which at least provides a useful alternative.

Other preferred objects of the present invention will become apparent from the following description.

### SUMMARY OF INVENTION

In one form, although it need not be the only or indeed the broadest form, there is provided a system for synchronising exercise or training measurement equipment, the system comprising:
an electronic device having a processor;
exercise equipment; and
a camera;
wherein the processor is configured to:
   receive measurement data from the exercise equipment;
   issue a request to the camera to start recording;
   determine a camera latency;
   calculate a latency synchronisation modifier using the determined camera latency and an exercise equipment communication latency between the electronic device and the exercise equipment; and
   synchronise the received measurement data from the exercise equipment with video data from the camera using the calculated latency synchronisation modifier.

The electronic device may comprises a display. The processor may be further configured to simultaneously output the synchronised received measurement data and video data to the display of the electronic device. The processor may be further configured to simultaneously output the synchronised received measurement data and video data to an external display.

The received measurement data may be displayed in the form of a graph. The graph may have a time axis. The video data may be navigated using an indicator that traverses the time axis of the graph. The indicator may comprise a vertical line that intersects the time axis of the graph. A user may navigate the video data by moving the indicator to points in time in the graph of measurement data.

The camera latency may initially be determined by calculating the delay the camera took to action the request to start recording. The camera may send an acknowledgement of request to the camera to start recording to the processor. The processor may receive an acknowledgement of request to the camera to start recording from the camera. The camera latency may be a computed delay between the issued request to the camera to start recording and the actual start of recording at the camera.

The exercise equipment communication latency may initially comprise a predetermined amount. The predetermined amount may correspond to an estimate of a latency for a communication protocol between the exercise equipment and the electronic device. The communication protocol between the exercise equipment and the electronic device may be wireless.

The wireless communication protocol between the exercise equipment and the electronic device may be Bluetooth. The predetermined amount may be between 5 ms and 100 ms, preferably between 10 ms and 50 ms, even more preferably between 20 and 40 ms. The processor may be further configured to update the exercise equipment communication latency to a calculated latency value. The processor may be further configured to update the exercise equipment communication latency to a calculated latency value if the device communication latency is determined to differ from the predetermined amount.

The measurement data from the exercise equipment may be captured at over 500 Hz, and preferably over 800 hz. In a preferred form the measurement data from the exercise equipment may be captured at 1 khz. The camera may capture the video data at over 100 frames per second, and preferably at at least 120 frames per second. The recording frequency of the measurement data from the exercise equipment and the frame rate of the video data from the camera may not be divisible.

The processor may be further configured to analyse the measurement data from the exercise equipment to automatically determine a type of exercise being performed. The processor may be further configured to analyse the measurement data from the exercise equipment to automatically determine and one or more points of interest. A determined point of interest in the measurement data from the exercise equipment may be matched to at least one frame in the video data. The frame in the video data that is matched to the point of interest in the measurement data may be tagged (or bookmarked). The tagged (or bookmarked) frame may be selectable by a user operating the electronic device. The processor may be configured to allow a sure to initiate video playback from the point of interest.

The electronic device may be a tablet. The camera may be integrated into the tablet. The exercise equipment may comprise a force platform. The exercise equipment may comprise strength testing equipment. The exercise equipment may comprise a speed gate.

Further features and advantages of the present invention will become apparent from the following detailed description.

### BRIEF DESCRIPTION OF THE DRAWINGS

By way of example only, preferred embodiments of the invention will be described more fully hereinafter with reference to the accompanying figures, wherein:

FIG. 1 is a diagrammatic view of a system for synchronising exercise or training measurement equipment;

FIG. 2 is a front view of a tablet showing simultaneous data display; and

FIG. 3 is a flow chart of a system for synchronising exercise or training measurement equipment at a processor.

### DETAILED DESCRIPTION OF THE DRAWINGS

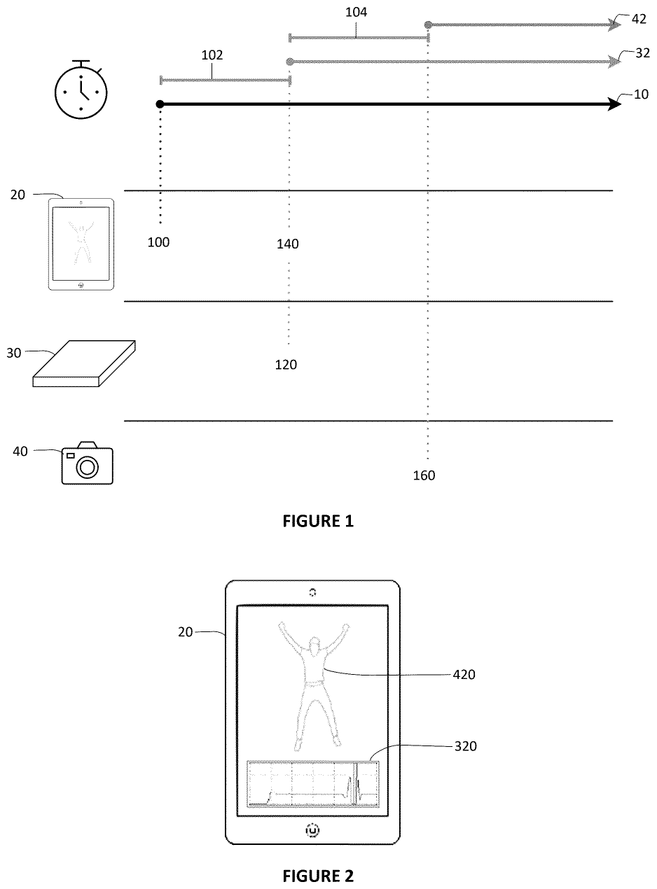

FIG. 1 illustrates a diagrammatic view of a system having a timeline 10, an electronic device in the form of a tablet 20, exercise equipment in the form of a force measurement platform 30, and a camera 40. Although the camera 40 is illustrated separately in the diagrammatic view of FIG. 1, it should be appreciated that it may be integrated into one of the other devices. In a preferred form the camera 40 is a camera of the tablet 20.

In use, a device such as the tablet 20 signals 100 a start to record active measurement on the exercise equipment 30. Due to communication latency 102, the signal 100 is received by the exercise equipment 30 a short time later at which point the exercise equipment 30 receives the request to recording force measurement data 120. At least initially, the device communication latency 102 may be assumed by a processor of the system to be a predetermined amount such as, for example, 30 ms for a Bluetooth communication connection. If the device communication latency 102 is subsequently determined to differ from the predetermined amount the processor can update its initial assumption to a calculated latency value. The processor is preferably a processor of the tablet 20, but it should be appreciated that the processor could be located in the exercise equipment 30 or be a further separate device. It should also be appreciated that more than one processor may be employed (e.g. one in the tablet 30 and one in the exercise equipment 30).

Once the exercise equipment 30 receives the request to record force measurement data 120 it sends an acknowledgement to the tablet 20 and starts measuring force measurement data 32. The tablet 20 then starts receiving data from the exercise equipment 30 and initiates a request to record video 140 with the camera 40. A camera latency 104 occurs between when the request to record video 140 is issued and the camera 40 actually starts recording 160. The camera latency 104 varies between individual recordings and the processor is configured to determine that latency. The camera latency 104 may be determined by comparing a timestamp difference between the time at request and the time at acknowledgement. The timestamps are preferably both captured using the same clock such as, for example, a system clock of the tablet 20.

Measurement data 32 from the exercise equipment 30 and video data 42 from the camera 40 are both recorded simultaneously on the tablet 20. As shown in FIG. 2, the force measurement data 32 may be displayed in a graph 320, or the like, simultaneously with a video feed 420 of the camera data 42. The graph 320 may form a progress bar with an indicator, such as a vertical line that traverses a time axis of the graph 320 during playback, that can be used to navigate the video feed 420. The force measurement data 32 and video data 42 are synchronised by applying a latency synchronisation modifier. This synchronisation preferably occurs on the tablet 20 but it should be appreciated this could be performed on one of the other devices or an external device such as a computer. The latency synchronisation modifier is calculated using both the exercise equipment communication latency 102 and camera latency 104 to accurately synchronise the force measurement data 32 from the exercise equipment 30 with the video data 42 from the camera 40.

The processor is configured to analyse the force measurement data 120 to automatically determine one or more of the type of exercise being performed and the points of interest of the exercise. The analysis of points of interest may, for example, identify the point in time in the force measurement data 32 that the subject exerted maximum force, lifted off the platform 30, landed on the platform 30, and the like. Such analysis is performed by interpreting the force measurement data 30 and comparing to known patterns. Both the type of exercise being performed and the points of interest of the exercise can be determined in this manner.

Once a point of interest has been determined, it is desirable to match this to a frame or set of frames in the video data 42. An issue that can arise in synchronising the force measurement data 32 with video data 42 is the difference in recording frequencies of each device. In preferred forms, the force measurement data 32 is captured at 1 kHz. A high framerate camera 40 used in the present invention may capture 120 or 240 frames per second. Notably, these numbers are not divisible so there are 8.333 or 4.167 force measurement data points per camera frame in either of these examples. The indivisibility of the two recording frequencies results in a small mismatch. In such circumstances a matching procedure may be to match datapoints that are nearest to each other after the latency synchronisation modifier has been applied. A frame that leads the point of calculated alignment may be selected to display or, in a preferred form, a selection of frames around the point of interest may be identified and grouped as relating to the point of interest. The selection of frames around the point of interest may be viewed as a video by a user. Depending upon timing this may result in a video data 42 frame that leads or lags an identified point of interest being matched, but unless a slow camera frame rate is used, which his less desirable for other reasons as well, this difference is largely imperceptible.

Once the points of interest and their matched frames have been identified, they may be tagged or bookmarked for easy identification and selection by a user. In preferred forms the tablet 20 runs an application having a graphical user interface (GUI) that can present the tagged points of interest to the user with a thumbnail image with automatically applied labels corresponding to the activity occurring at that identified point of interest.

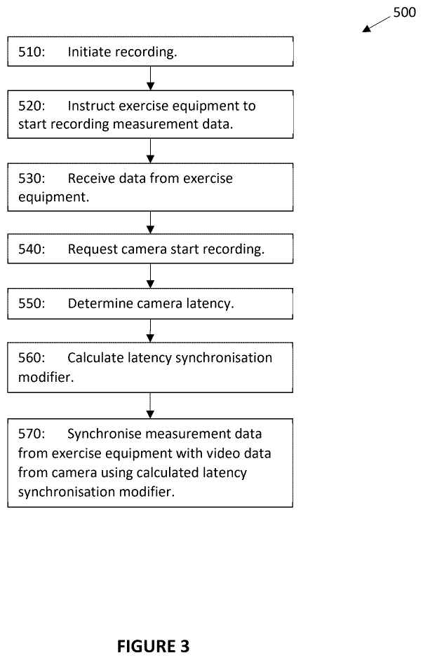

FIG. 3 illustrates a flowchart 500 of steps the processor may undertake to synchronise exercise or training measurement equipment (such as, for example, a tablet 20, camera 40, and force platform 30). At step 510 the processor first receives an instruction to initiate a recording. The instruction may come from a user or could be triggered by some other event such as, for example, a subject interacting with a piece of exercise equipment (e.g. the subject stepping onto a force platform 30).

At step 520 the processor then instructs the exercise equipment, such as a force platform 30, to start recording measurement data. After a short period of time, due at least in part to device communication latency 102, the processor receives measurement data 32 from the exercise equipment at step 530. At step 540 the processor requests the camera 40 to start recording and then at step 550 the processor computes a camera latency 104. The camera latency 104 is between the request 540 being sent when the camera 40 actually starts recording which, in the case of the camera 40 and processor both being located on the tablet 20, can be measured directly by the processor using a system clock.

At step 560 the processor calculates a latency synchronisation modifier. The latency synchronisation modifier is calculated using an exercise equipment communication latency 102 and the camera latency calculated at step 550. The latency synchronisation modifier can then be used by the processor, at step 570, to synchronise measurement data from exercise equipment with video data from camera.

Advantageously, the present invention allows exercise equipment measurement data to be automatically and accurately synchronised and aligned with video data. The determination of points of interest from either the exercise equipment measurement data or video data enables a user to jump quickly and directly to specific events and see what was occurring with the subject at, and around, that point in time. As the determination of points of interest is automatic, the user can very quickly identify and review the measurement and/or video data associated with that point of interest quickly. A short video of the point of interest, which may include some frames before and after, can be easily selected and played by a user to avoid the need to scroll back and forth through the video and/or data trying which is not only time consuming but less accurate and repeatable.

Although the invention is described primarily with synchronising force plate measurements, it should be appreciated that the principals can be applied to other types of exercise or training equipment such as, for example, strength testing equipment, speed gates, and the like. It should also be appreciated that an exercise may be any human movement for fitness, diagnostics, rehabilitation, biomechanics, human kinematics, or the like, and no limitation is meant thereby.

In this specification, adjectives such as first and second, left and right, top and bottom, and the like may be used solely to distinguish one element or action from another element or action without necessarily requiring or implying any actual such relationship or order. Where the context permits, reference to an integer or a component or step (or the like) is not to be interpreted as being limited to only one of that integer, component, or step, but rather could be one or more of that integer, component, or step etc.

The above description of various embodiments of the present invention is provided for purposes of description to one of ordinary skill in the related art. It is not intended to be exhaustive or to limit the invention to a single disclosed embodiment. As mentioned above, numerous alternatives and variations to the present invention will be apparent to those skilled in the art of the above teaching. Accordingly, while some alternative embodiments have been discussed specifically, other embodiments will be apparent or relatively easily developed by those of ordinary skill in the art. The invention is intended to embrace all alternatives, modifications, and variations of the present invention that have been discussed herein, and other embodiments that fall within the spirit and scope of the above described invention.

As used herein, an element or operation recited in the singular and preceded with the word “a” or “an” should be understood as not excluding plural elements or operations, unless such exclusion is explicitly recited. Furthermore, references to “one embodiment” of the present disclosure are not intended to be interpreted as excluding the existence of additional embodiments that also incorporate the recited features.

In this specification, the terms ‘comprises’, ‘comprising’, ‘includes’, ‘including’, or similar terms are intended to mean a non-exclusive inclusion, such that a method, system or apparatus that comprises a list of elements does not include those elements solely, but may well include other elements not listed.

1. A system for synchronising exercise or training measurement equipment, the system comprising:
an electronic device having a processor;
exercise equipment; and
a camera;
wherein the processor is configured to:
receive measurement data from the exercise equipment;
issue a request to the camera to start recording;
determine a camera latency;
calculate a latency synchronisation modifier using the determined camera latency and an exercise equipment communication latency between the electronic device and the exercise equipment; and
synchronise the received measurement data from the exercise equipment with video data from the camera using the calculated latency synchronisation modifier.

2. The system of claim 1, wherein the electronic device comprises a display and the processor is further configured to simultaneously output the synchronised received measurement data and video data to that display.

3. The system of claim 2, wherein the received measurement data is displayed in the form of a graph with a time axis and the video data may be navigated using an indicator that traverses the time axis of the graph.

4. The system of any one of claims 1 to 3, wherein the camera latency is at least initially determined by calculating the delay the camera took to action the request to start recording.

5. The system of claim 1, wherein the exercise equipment communication latency is initially a predetermined amount that corresponds to an estimate of a latency for a communication protocol between the exercise equipment and electronic device.

6. The system of claim 5, wherein the predetermined amount is between 5 ms and 100 ms.

7. The system of claim 5, wherein the processor updates its exercise equipment communication latency to a calculated latency value if the device communication latency is determined to differ from the predetermined amount.

8. The system of claim 1, wherein the measurement data from the exercise equipment is captured at over 500 Hz.

9. The system of claim 1, wherein the camera captures video data at over 100 frames per second.

10. The system of claim 1, wherein a recording frequency of the measurement data from the exercise equipment and a frame rate of the video data from the camera are not divisible.

11. The system of claim 1, wherein the processor is further configured to analyse the measurement data from the exercise equipment to automatically determine a type of exercise being performed.

12. The system of claim 1, wherein the processor is further configured to analyse the measurement data from the exercise equipment to automatically determine and one or more points of interest.

13. The system of claim 12, wherein a determined point of interest in the measurement data from the exercise equipment is matched to at least one frame in the video data.

14. The system of claim 13 wherein the at least one frame in the video data that is matched to the point of interest in the measurement data is tagged and can be selectable by a user operating the electronic device.

15. The system of claim 14, wherein a video of frames from the video data around the point of interest in the measurement data is played upon selection by the user.

16. The system of claim 1, wherein the electronic device is a tablet and the camera is integrated into the tablet.

17. The system of claim 1 wherein the exercise equipment comprises one or more of a force platform, strength testing equipment, or a speed gate.
